(19) United States
(12) Patent Application Publication
(10) Pub. No.: US 2008/0033275 A1
(43) Pub. Date: Feb. 7, 2008

(54) Method and Apparatus for Sample Probe Movement Control

(76) Inventors: Thomas B. Blank, Gilbert, AZ (US); Alan Abul-Haj, Mesa, AZ (US); Timothy W. Stippick, Phoenix, AZ (US); James Ryan Henderson, Phoenix, AZ (US); Christopher Slawinski, Mesa, AZ (US); David W. Leake, Peoria, AZ (US)

Correspondence Address: GLENN PATENT GROUP, Suite L, 3475 Edison Way, Menlo Park, CA, 94025, US

(21) Appl. No.: 11/625,752

(22) Filed: Jan. 22, 2007

Related U.S. Application Data
(63) Continuation-in-part of application No. 11/117,104, filed on Apr. 27, 2005.
(60) Provisional application No. 60/566,568, filed on Apr. 28, 2004.
(60) Provisional application No. 60/864,375, filed on Nov. 3, 2006.
(60) Provisional application No. 60/761,486, filed on Jan. 23, 2006.

(51) Int. Cl. A61B 5/00

(52) U.S. Cl. 600/365

(57) ABSTRACT

A prove interface method and apparatus for an optical based noninvasive analyzer includes an algorithm for controlling placement and/or orientation of the analyzer sample probe relative to a sample site in a dynamic and/or static fashion. The sample probe tip relative to a sample site is controlled with respect to any one or more of: x-axis position, y-axis position, z-axis position, rotational orientation, and tilt.

### CROSS REFERENCE TO RELATED APPLICATIONS

This application is a continuation-in-part of:
U.S. patent application Ser. No. 11/117,104, filed Apr. 27, 2005, which claims the benefit of U.S. provisional application No. 60/566,568, filed Apr. 28, 2004;
   and claims benefit of:
U.S. provisional patent application Ser. No. 60/864,375 filed Nov. 3, 2006; and of
U.S. provisional patent application Ser. No. 60/761,486 filed Jan. 23, 2006;
   each of which are incorporated herein in its entirety by this reference thereto.

### BACKGROUND OF THE INVENTION

1. Field of the Invention

This invention relates generally to measurement of glucose concentration in tissue, and more particularly to sample probe movement control in the measurement of glucose concentration in tissue.

2. Discussion of the Related Art

Sampling deformable skin tissue with a spectrometer is complicated by optical and mechanical mechanisms occurring before and/or during sampling.

In a first case, a representative optical sample of an object is collected without contacting the object with the spectrometer. In this case, specular reflectance and stray light is of concern. In one instance, mechano-optical methods are used to reduce the amount of specularly reflected light collected. However, this is greatly complicated by an object having a surface that diffusely scatters light. In a second instance, an algorithm is used to reduce the effects of specular reflectance. This is complicated by specularly reflected light contributing in an additive manner to the resultant spectrum. The additive contribution results in a nonlinear interference, which results in a distortion of the spectrum that is difficult to remove. The problem is greatly enhanced as the magnitude of the analyte signal decreases. Thus, for low signal-to-noise ratio measurements, specularly reflected light is preferably avoided. For example, noninvasively determining an analyte property, such as glucose concentration, from a spectrum of the body is complicated by additive specularly reflected light in the collected spectrum. As the analyte signal decreases in magnitude, the impact of specularly reflected light increases.

In a second case, a spectrum of an object is collected after contacting the object with a spectrometer. For objects or samples that are deformable, the optical properties of the sample are changed due to contact of an optical probe with the sample, which deforms the sample and results in changed optical properties of the sample. Changed optical properties due to movement of a sample before or during sampling include:
absorbance; and
scattering.

In this second case, the sampling method alters the sample, often detrimentally. The changes in the sample resulting from the sampling method degrade resulting sample interpretation. As the signal level of the analyte decreases, the relative changes in the sample due to sampling result in increasing difficulty in extraction of analyte signal. In some instances, the sampling induced changes preclude precise and/or accurate analyte property determination from a sample spectrum. For example, a sample probe contacting skin of a human alters the sample. Changes to the skin sample upon contact, during sampling, and/or before sampling include:
stretching of skin;
compression of skin; and
altered spatial distribution of sample constituents.

Further, the changes are often time dependent and methodology of sampling dependent. Typically, the degree of contact to the sample by the spectrometer results in nonlinear changes to a resulting collected spectrum. Manually manipulating a spectrometer during the method of optical sampling requires human interaction. Humans are limited in terms of dexterity, precision, reproducibility, and sight. For example, placing a spectrometer in contact with an object during sampling is complicated by a number of parameters including any of:
not being able to reach and see the sample at the same time;
the actual sampling area being visually obscured by part of the spectrometer or sample;
placing the analyzer relative to the sample within precision and/or accuracy specifications at, near, or beyond human control limits; and
repeatedly making a measurement due to human fatigue and frailty.
   Noninvasive Technologies

There are a number of reports on noninvasive technologies. Some of these relate to general instrumentation configurations, such as those required for noninvasive glucose concentration estimation, while others refer to sampling technologies. Those related to the present invention are briefly reviewed here:

P. Rolfe, Investigating substances in a patient's bloodstream, U.K. patent application ser. no. 2,033,575 (Aug. 24, 1979) describes an apparatus for directing light into the body, detecting attenuated backscattered light, and using the collected signal to determine glucose concentrations in or near the bloodstream.

C. Dahne, D. Gross, Spectrophotometric method and apparatus for the non-invasive, U.S. Pat. No. 4,655,225 (Apr. 7, 1987) describe a method and apparatus for directing light into a patient's body, collecting transmitted or backscattered light, and determining glucose concentrations from selected near-infrared wavelength bands. Wavelengths include 1560 to 1590, 1750 to 1780, 2085 to 2115, and 2255 to 2285 nm with at least one additional reference signal from 1000 to 2700 nm.

R. Barnes, J. Brasch, D. Purdy, W. Lougheed, Non-invasive determination of analyte concentration in body of mammals, U.S. Pat. No. 5,379,764 (Jan. 10, 1995) describe a noninvasive glucose concentration estimation analyzer that uses data pretreatment in conjunction with a multivariate analysis to estimate blood glucose concentrations.

M. Robinson, K. Ward, R. Eaton, D. Haaland, Method and apparatus for determining the similarity of a biological analyte from a model constructed from known biological fluids, U.S. Pat. No. 4,975,581 (Dec. 4, 1990) describe a method and apparatus for measuring a concentration of a biological analyte, such as glucose concentration, using infrared spectroscopy in conjunction with a multivariate model. The multivariate model is constructed from a plurality of known biological fluid samples.

J. Hall, T. Cadell, Method and device for measuring concentration levels of blood constituents non-invasively, U.S. Pat. No. 5,361,758 (Nov. 8, 1994) describe a noninvasive device and method for determining analyte concentrations within a living subject using polychromatic light, a wavelength separation device, and an array detector. The apparatus uses a receptor shaped to accept a fingertip with means for blocking extraneous light.

S. Malin, G Khalil, Method and apparatus for multi-spectral analysis of organic blood analytes in noninvasive infrared spectroscopy, U.S. Pat. No. 6,040,578 (Mar. 21, 2000) describe a method and apparatus for determination of an organic blood analyte using multi-spectral analysis in the near-infrared. A plurality of distinct nonoverlapping regions of wavelengths are incident upon a sample surface, diffusely reflected radiation is collected, and the analyte concentration is determined via chemometric techniques.

Specular Reflectance

R. Messerschmidt, D. Sting Blocker device for eliminating specular reflectance from a diffuse reflectance spectrum, U.S. Pat. No. 4,661,706 (Apr. 28, 1987) describe a reduction of specular reflectance by a mechanical device. A blade-like device “skims” the specular light before it impinges on the detector. A disadvantage of this system is that it does not efficiently collect diffusely reflected light and the alignment is problematic.

R. Messerschmidt, M. Robinson Diffuse reflectance monitoring apparatus, U.S. Pat. No. 5,636,633 (Jun. 10, 1997) describe a specular control device for diffuse reflectance spectroscopy using a group of reflecting and open sections.

R. Messerschmidt, M. Robinson Diffuse reflectance monitoring apparatus, U.S. Pat. No. 5,935,062 (Aug. 10, 1999) and R. Messerschmidt, M. Robinson Diffuse reflectance monitoring apparatus, U.S. Pat. No. 6,230,034 (May 8, 2001) describe a diffuse reflectance control device that discriminates between diffusely reflected light that is reflected from selected depths. This control device additionally acts as a blocker to prevent specularly reflected light from reaching the detector.

Malin, supra, describes the use of specularly reflected light in regions of high water absorbance, such as 1450 and 1900 nm, to mark the presence of outlier spectra wherein the specularly reflected light is not sufficiently reduced.

K. Hazen, G. Acosta, A. Abul-Haj, R. Abul-Haj, Apparatus and method for reproducibly modifying localized absorption and scattering coefficients at a tissue measurement site during optical sampling, U.S. Pat. No. 6,534,012 (Mar. 18, 2003) describe a mechanical device for applying sufficient and reproducible contact of the apparatus to the sampling medium to minimize specular reflectance. Further, the apparatus allows for reproducible applied pressure to the sample site and reproducible temperature at the sample site.

Temperature

K. Hazen, Glucose Determination in Biological Matrices Using Near-Infrared Spectroscopy, doctoral dissertation, University of Iowa (1995) describes the adverse effect of temperature on near-infrared based glucose concentration estimations. Physiological constituents have near-infrared absorbance spectra that are sensitive, in terms of magnitude and location, to localized temperature and the sensitivity impacts noninvasive glucose concentration estimation.

Pressure

E. Chan, B. Sorg, D. Protsenko, M. O'Neil, M. Motamedi, A. Welch, Effects of compression on soft tissue optical properties, IEEE Journal of Selected Topics in Quantum Electronics, Vol. 2, no. 4, pp. 943-950 (1996) describe the effect of pressure on absorption and reduced scattering coefficients from 400 to 1800 nm. Most specimens show an increase in the scattering coefficient with compression.

K. Hazen, G. Acosta, A. Abul-Haj, R. Abul-Haj, Apparatus and method for reproducibly modifying localized absorption and scattering coefficients at a tissue measurement site during optical sampling, U.S. Pat. No. 6,534,012 (Mar. 18, 2003) describe in a first embodiment a noninvasive glucose concentration estimation apparatus for either varying the pressure applied to a sample site or maintaining a constant pressure on a sample site in a controlled and reproducible manner by moving a sample probe along the z-axis perpendicular to the sample site surface. In an additional described embodiment, the arm sample site platform is moved along the z-axis that is perpendicular to the plane defined by the sample surface by raising or lowering the sample holder platform relative to the analyzer probe tip. The '012 patent further teaches proper contact to be the moment specularly reflected light is about zero at the water bands about 1950 and 2500 nm.

Coupling Fluid

A number of sources describe coupling fluids with important sampling parameters.

Index of refraction matching between the sampling apparatus and sampled medium to enhance optical throughput is known. Glycerol is a common index matching fluid for optics to skin.

R. Messerschmidt, Method for non-invasive blood analyte measurement with improved optical interface, U.S. Pat. No. 5,655,530 (Aug. 12, 1997), and R. Messerschmidt Method for non-invasive blood analyte measurement with improved optical interface, U.S. Pat. No. 5,823,951 (Oct. 20, 1998) describe an index-matching medium for use between a sensor probe and the skin surface. The index-matching medium is a composition containing both perfluorocarbons and chlorofluorocarbons.

M. Robinson, R. Messerschmidt, Method for non-invasive blood analyte measurement with improved optical interface, U.S. Pat. No. 6,152,876 (Nov. 28, 2000) and M. Rohrscheib, C. Gardner, M. Robinson, Method and apparatus for non-invasive blood analyte measurement with fluid compartment equilibration, U.S. Pat. No. 6,240,306 (May 29, 2001) describe an index-matching medium to improve the interface between the sensor probe and skin surface during spectroscopic analysis. The index-matching medium is preferably a composition containing chlorofluorocarbons with optional added perfluorocarbons.

T. Blank, G. Acosta, M. Mattu, S. Monfre, Fiber optic probe guide placement guide, U.S. Pat. No. 6,415,167 (Jul. 2, 2002) describe a coupling fluid of one or more perfluoro compounds where a quantity of the coupling fluid is placed at an interface of the optical probe and measurement site. Perfluoro compounds do not have the toxicity associated with chlorofluorocarbons.

M. Makarewicz, M. Mattu, T. Blank, G. Acosta, E. Handy, W. Hay, T. Stippick, B. Richie, Method and apparatus for minimizing spectral interference due to within and between sample variations during in-situ spectral sampling of tissue, U.S. patent application Ser. No. 09/954,856 (filed Sep. 17, 2001) describe a temperature and pressure controlled sample interface. The means of pressure control are a set of supports for the sample that control the natural position of the sample probe relative to the sample.

Positioning

E. Ashibe, Measuring condition setting jig, measuring condition setting method and biological measuring system, U.S. Pat. No. 6,381,489, Apr. 30, 2002 describes a measurement condition setting fixture secured to a measurement site, such as a living body, prior to measurement. At time of measurement, a light irradiating section and light receiving section of a measuring optical system are attached to the setting fixture to attach the measurement site to the optical system.

J. Röper, D. Böcker, System and method for the determination of tissue properties, U.S. Pat. No. 5,879,373 (Mar. 9, 1999) describe a device for reproducibly attaching a measuring device to a tissue surface.

J. Griffith, P. Cooper, T. Barker, Method and apparatus for non-invasive blood glucose sensing, U.S. Pat. No. 6,088,605 (Jul. 11, 2000) describe an analyzer with a patient forearm interface in which the forearm of the patient is moved in an incremental manner along the longitudinal axis of the patient's forearm. Spectra collected at incremental distances are averaged to take into account variations in the biological components of the skin. Between measurements rollers are used to raise the arm, move the arm relative to the apparatus and lower the arm by disengaging a solenoid causing the skin lifting mechanism to lower the arm into a new contact with the sensor head.

T. Blank, G. Acosta, M. Mattu, S. Monfre, Fiber optic probe placement guide, U.S. Pat. No. 6,415,167 (Jul. 2, 2002) describe a coupling fluid and the use of a guide in conjunction with a noninvasive glucose concentration analyzer in order to increase precision of the location of the sampled tissue site resulting in increased accuracy and precision in noninvasive glucose concentration estimations.

T. Blank, G. Acosta, M. Mattu, M. Makarewicz, S. Monfre, A. Lorenz, T. Ruchti, Optical sampling interface system for in-vivo measurement of tissue, world patent publication no. WO 2003/105664 (filed Jun. 11, 2003) describe an optical sampling interface system that includes an optical probe placement guide, a means for stabilizing the sampled tissue, and an optical coupler for repeatedly sampling a tissue measurement site in-vivo.

Targeting

G. Lucassen, G. Puppels, P. Caspers, M. Van Der Voort, E. Lenderink, M. Van Der Mark, R. Hendricks, J. Cohen, Analysis of a composition, U.S. Pat. No. 6,609,015 (Aug. 19, 2003); G. Lucassen, R. Hendricks, M. Van Der Voort, G. Puppels, Analysis of a composition, U.S. Pat. No. 6,687,520 (Feb. 3, 2004); G. Lucassen, G. Puppels, M. Van Der Voort, Analysis Apparatus and Method, WIPO publication no. WO 2004/058058 (filed Dec. 4, 2003); F. Schuurmans, M. Van Beek, L. Bakker, W. Rensen, B. Hendricks, R. Hendricks, T. Steffen, Optical analysis system, WIPO publication no. WO 2004/057285 (filed Dec. 19, 2003); G. Lucassen, G. Puppels, M. Van Der Voort, R. Wolthuis, Apparatus and method for blood analysis, WIPO publication no. WO 2004/070368 (filed Jan. 19, 2004); R. Hendricks, G. Lucassen, M. Van Der Voort, G. Puppels, M. Van Beek, Analysis of a composition with monitoring, WIPO publication no. WO 2004/082474 (filed Mar. 15, 2004); M. Van Beek, C. Liedenbaum, G. Lucassen, W. Rensen Catheter head, WIPO publication no. WO 2004/093669 (filed Apr. 23, 2004); and M. Van Beek, J. Horsten, M. Van Der Voort, G. Lucassen, P. Caspers, Method and apparatus for determining a property of a fluid which flows through a biological tubular structure with variable numerical aperture, WIPO publication no. WO 2005/009236 (filed Jul. 26, 2004) describe a monitoring (targeting) system used to direct a Raman excitation system to a blood vessel.

The Raman spectroscopy targeting and imaging systems described above are fundamentally different than the vibrational absorption spectroscopy techniques taught herein. Raman spectra are obtained by irradiating a sample to a high energy state with a powerful source of visible monochromatic radiation. In addition, Raman spectroscopy is strongly dependent upon size of scattering particles. Finally, Raman signals require a change in polarizability of the probed molecule for a signal to be obtained. By contrast, infrared spectrometers use notably less intense broadband sources that do not excite molecules to high energy states. Further, infrared absorption spectroscopy in the region 1300 to 2500 nm is relatively insensitive to particle size. Finally, molecules that are vibrationally active require a change in dipole, which is associated with the vibrational mode of the molecule. This last point is critical. Raman signals require a change in polarizability while vibrational spectroscopy requires a change in dipole. This means molecular structure that is vibrationally infrared active is inactive in Raman spectroscopy and vise-versa. For example, near-infrared and infrared vibrational absorption spectroscopy show water to have very strong absorbance signal resulting in the primary interference in tissue analysis. By contrast, Raman signals are virtually unperturbed by water. As a result, in absorption spectroscopy regions of high water absorbance are necessarily avoided and penetration depths of photons into tissue from 1100 to 2500 nm is limited to millimeters. By contrast, Raman signals are possible in regions where water absorbs strongly in the infrared and a greater depth of penetration of photons into tissue is possible. Therefore, Raman and vibrational infrared spectroscopy operate under different theory, use different instrumentation, observe different molecular structure, and sample different tissue layers in skin.

There exists a need for controlling optical based sampling methods to minimize collection of specularly reflected light, for minimizing collection of stray light, and for minimizing sampling related changes to a deformable sample. For optical sampling of a deformable object, it would be desirable to provide a method and apparatus that automatically reduces the effects of non-contact and excessive contact of the sample during sampling.

### SUMMARY OF THE INVENTION

The invention relates generally to a probe interface method and apparatus for use in conjunction with a noninvasive analyzer. More particularly, an algorithm controls a sample probe placement relative to a sample site in a dynamic or static fashion.

### BRIEF DESCRIPTION OF THE DRAWINGS

FIG. 1A is a block schematic diagram of a tissue probe sensor active control system.

FIG. 1B is a block schematic diagram of various converters useful in the actuator system of the probe sensor control system of FIG. 1A.

FIG. 2 is a graph of change in tissue absorbance as a function of displacement;

FIG. 3 is a graph of changing absorbance related to chemical features as a result of tissue displacement;

FIG. 4 is a graph of changing absorbance related to chemical features as a result of tissue displacement;

FIG. 5 is a perspective cross-section graphical illustration of skin features affected by displacement of skin by a sample probe;

FIG. 6 is a block diagram of a noninvasive analyzer;

FIG. 7A is a side cross-sectional view of a sample module having a sample probe having capacitance sensors.

FIG. 7B is an end plan view of the sample module of FIG. 7A.

FIG. 8 is a schematic diagram of a circuit for measuring a time constant;

FIG. 9 is a schematic diagram of an amplifier circuit for measuring a time constant;

FIGS. 10A, 10B, 10C and 10D are end plan views of various capacitance sensor layouts for use with sample probes;

FIG. 11 is a cross-sectional view of an exemplary sample probe;

FIG. 12 is an electrical schematic diagram of a conductive contact system;

FIG. 13 is a flow diagram of a qualification/monitoring system;

FIG. 14 is a graph of exemplary contact response data as a function of time/position;

FIG. 15 is a graph of exemplary contact response data as a function of time/position;

FIG. 16 is a graph of exemplary contact response data as a function of time/position;

FIG. 17 is a block schematic diagram of an example of an apparatus for using light in distance and/or tilt positioning of sample probe; and

FIG. 18 is a block schematic diagram of a multiple sensor positioning system.

### DETAILED DESCRIPTION OF THE INVENTION

An optical based noninvasive analyzer is provided with an algorithm that controls an analyzer sample probe placement and/or orientation relative to a sample site in a dynamic and/or static fashion, for measuring glucose concentration in tissue. In various embodiments, the sample probe tip relative to a sample site is controlled with respect to one or more of:
x-axis position;
y-axis position;
z-axis position;
rotational orientation; and
tilt.

Precise and/or accurate positioning of a sample probe tip with a sample site is beneficial to analyte property determination. For example, x- and y-axis positioning is used to sample the same or nominally the same location on a sample. As a further example, z-axis positioning is used to position the probes to ensure minimal collection of spectrally reflected light and/or to provide any of:
proximate contact of a sample probe tip with a sample site;
contact of a sample probe tip with a sample site; and
displacement of a sample probe tip into a deformable sample site.

Tilt control is used to prevent excessive skin stretch when a flat surface, such as a sample probe tip or a guide, is brought into contact with a deformable sample site, such as tissue where tissue is often irregular and has generally non-flat tissue surface topology. Tilt control allows the sensing portion of a sample probe, such as a center of an optical probe tip, to be brought into contact with a sample site without displacement and hence stretching of nearby skin by the edges of the optical probe as the sample probe tip is brought into contact at an angle normal to the irregular sample surface.

Herein, methods and apparatus for estimating glucose concentration from noninvasive spectra are used as a specific example of the invention.

Coordinate System

Herein, an x-, y-, and z-axes coordinate system relative to a given body part is defined. An x-, y-, z-coordinate system is used to define the sample site, movement of objects about the sample site, changes in the sample site, and physical interactions with the sample site. The x-axis is defined along the length of a body part and the y-axis is defined across the body part. As an illustrative example using a sample site on the forearm, the x-axis runs between the elbow and the wrist and the y-axis runs across the axis of the forearm. Similarly, for a sample site on a digit of the hand, the x-axis runs between the base and tip of the digit and the y-axis runs across the digit. Together, the x-, y-plane tangentially touches the skin surface, such as at a sample site. The z-axis is defined as orthogonal to the plane defined by the x- and y-axis. For example, a sample site on the forearm is defined by an x-, y-plane tangential to the sample site. An object, such as a sample probe, moving along an axis perpendicular to the x-, y-plane is moving along the z-axis. Rotation of an object about one or a combination of axes is further used to define the orientation of an object, such as a sample probe, relative to the sample site. Tilt refers to an off z-axis alignment of the longitudinal orientation of the sample probe where the longitudinal axis extends from the sample probe tip interfacing with a sample site to the opposite end of the sample probe.

Instrumentation

Herein, an analyzer comprises at least a source coupled via optics to a detector. In one embodiment, the analyzer is handheld. In a second embodiment, the analyzer sits upon a supporting surface during use. In a third embodiment, the analyzer is split having a base module physically separated from the sample module, where the sample module interfaces with the sample site during operational use. In the third embodiment, the base module is connected to the sample module via wireless communication, is connected via a communication bundle, or is connected through a semi-rigid weight support system, where the weight support system serves to support at least a portion of the weight of the sample module during use. The weight support system preferably operates as an automated positioning system or an actuation system. In the third embodiment, the weight support system carries any of:
optical signal;
data signal;
power;
electrical control signal; and
a fluid between the base module and the sample module.

In the third embodiment, the analyzer is preferably a split analyzer where the weight support system additionally operates as a communication bundle.

Any of the systems described herein are operable in a home environment, public facility, or in a medical environment, such as an emergency room, critical care facility, intensive care unit, hospital room, or medical professional patient treatment area. For example, the split analyzer is operable in a critical care facility where the sample module is positioned in proximate contact with a subject or patient during use and where the base module is positioned on a support surface, such as a rack, medical instrumentation rack, table, or wall mount. Optical components, such as a source, backreflector, guiding optics, lenses, filters, mirrors, a wavelength separation device, and at least one detector are optionally positioned in the base module and/or sample module.

In one illustrative system, at least a portion of the sample module is movable with respect to a subject or patient sample site along any of the x-, y-, and z-axes, and/or in terms of rotation or tilt. For example, the sample site, such as a forearm remains stationary while at least a portion of the analyzer, such as the sample probe head, moves to interface with the sample in terms of an x-, y-, and/or z-axis and/or with respect to rotation or tilt.

In another illustrative system, the components defining the optical train of an analyzer are moved as a unit to reposition the sample probe tip relative to a sample site. Moving the optical units of an analyzer together allows for fixed optics. For instance moving the sample probe head to the sample does not change the collection optic pathway so that collection optics are spatially fixed or controlled in terms of optically coupling the tip of the fiber bundle to the detection element. In one case, optics in the optical train from the sample to the detector are moved together in a controlled fashion.

In yet another illustrative system, the analyzer is held in a fixed position while the sample site is moved relative to the analyzer by moving a body part, such as with an adjustable platform, along any of the x-, y-, and z-axes, and/or in terms of rotation or tilt.

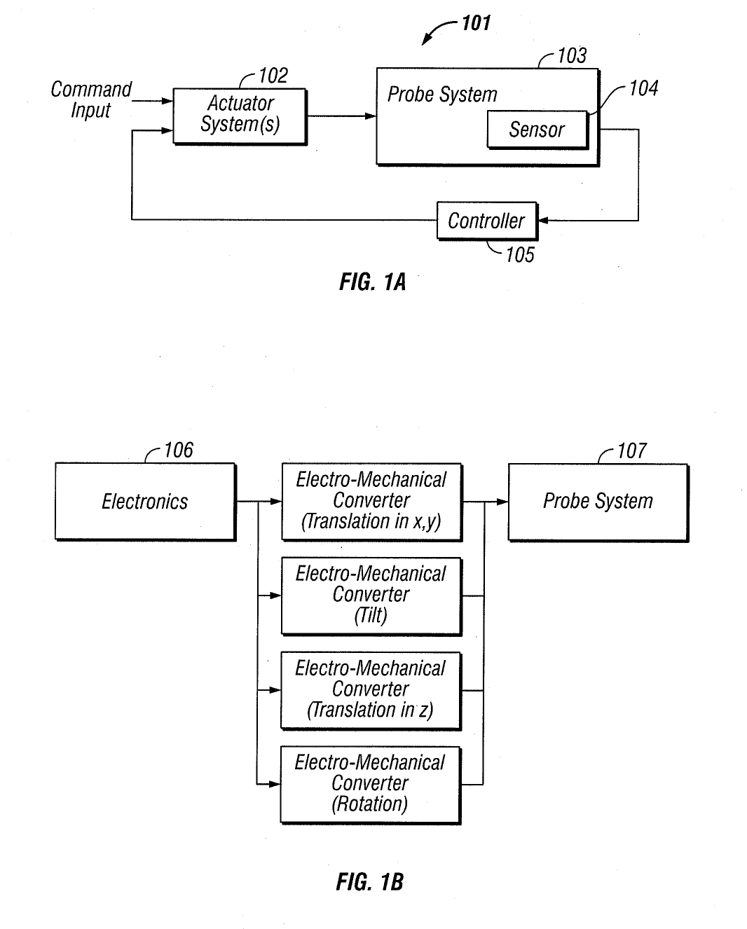

In the systems described herein, the movable portion of the sample module may move in an active or passive manner. When the movable portion of the sample module moves in an active manner, a controller is preferably used to position the tip of the sample probe relative to a tissue sample site, as presented in FIG. 1. Referring now to FIG. 1A, a command input is sent to an actuator system 102 within the analyzer 101, where the actuator system is preferably in the sample module. The actuator system 102 controls the position of a probe system having a sensor. The position is relative to a sample site, such as a tissue sample site. The signal from the sensor is either used in the measurement of an analyte property value of the sample or is used as input for a controller 105 that sends additional input to the actuator system to further control position or movement of the probe system via the actuator system. Referring now to FIG. 1B, the actuator system 102 preferably contains electronics 106 used in conjunction with electromechanical conversion of the movable aspect of the analyzer 101, such as the tip of the probe system 107 within the sample module in terms of one or more of: x-position, y-position, z-position, tilt, and rotation of the sample probe tip relative to the sample site.

Effect of Displacement on Tissue Spectra

Importance of control of the tip of the sample probe relative to a tissue site is herein demonstrated by example using noninvasive near-infrared spectra of tissue.

In a first example, the effect of displacement of a sample probe into a tissue sample is demonstrated. A movable sample probe at least partially contained in a sample module was moved from a first position not in contact with the sample to a position of displacement of a tissue sample through a series of steps.

In this example, the movable portion of the sample probe was guided to the sample location with an optional guide element described in T. Blank, G. Acosta, M. Mattu, S. Monfre, Fiber optic probe guide placement guide, U.S. Pat. No. 6,415,167 issued Jul. 2, 2002, which is incorporated herein in its entirety by this reference thereto. The guide element is replaceably attached to the sample site. The attachment of the guide to the sample site results in formation of a meniscus of skin in the opening of the guide. The meniscus is typically a convex bulge of tissue from the nominal plane of the skin tissue but is flat or concave in some individuals, such as older individuals or people with less collagen density at the sample site. The size of the meniscus is subject dependent, varies on a given subject from day-to-day, and varies on a subject within a day. In this experiment, a series of spacers were placed on top of the guide that sterically provide a stop to the sample probe as the sample probe moves down the z-axis, perpendicular to the skin surface, toward the tissue sample. As individual spacers are removed, the sample probe initiates contact with the sample. Removal of additional spacers results in probe displacement of the deformable tissue sample.

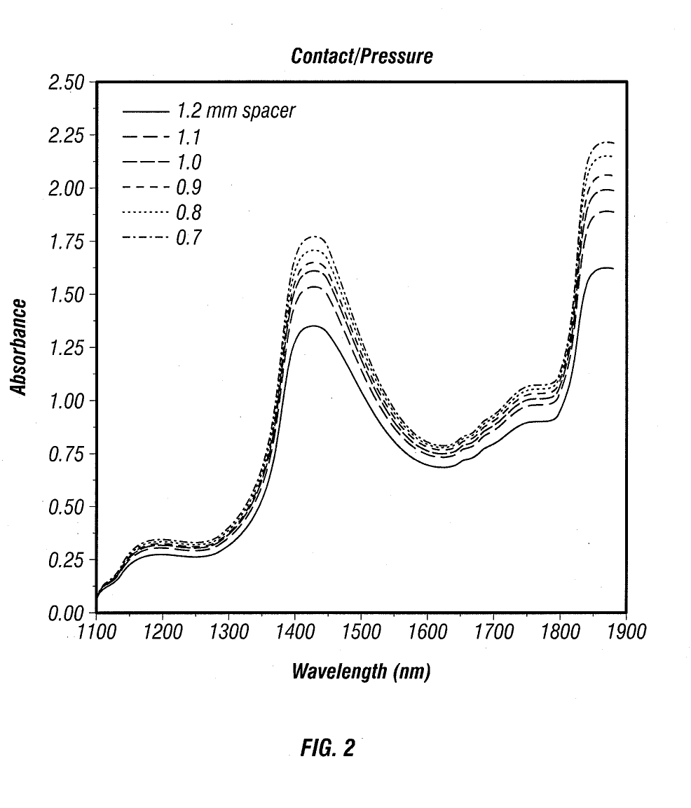

Spectra were collected with subsequent removal of the steric stops described, supra. The resulting spectra from 1100 to 1930 nm, collected with a 1.2, 1.1, 1.0, 0.9, 0.8, and 0.7 mm spacer after conversion to absorbance, are presented in FIG. 2.

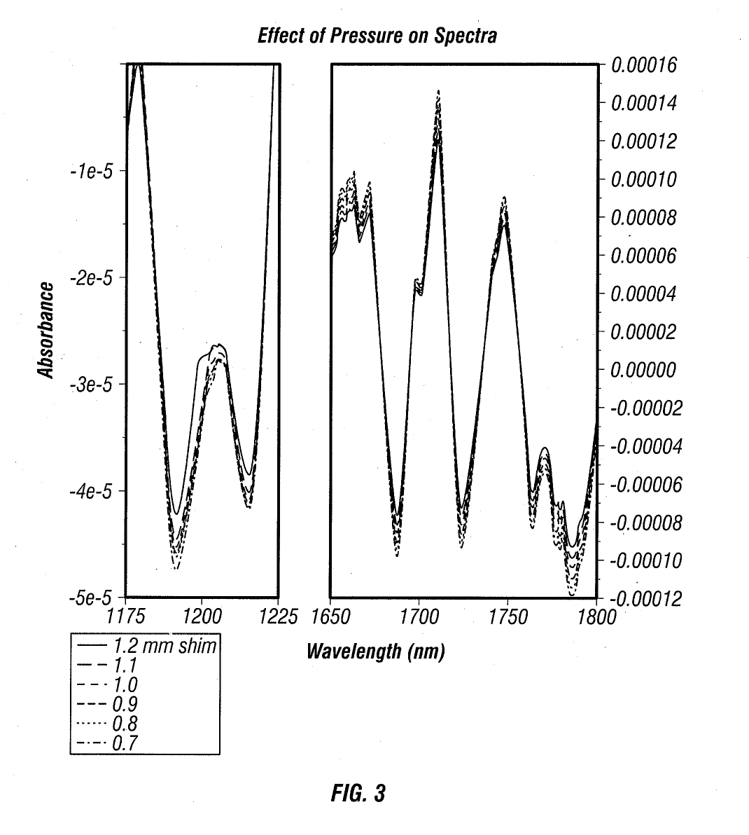

It is the relative movement of the sample probe along the z-axis relative to the tissue sample that is important as opposed to the size of the spacers. The observed intensity decreases, increases in absorbance, as spacers are removed and contact followed by displacement of the tissue results. Two dominant spectral features are observed: the light of the second overtone region from 1100 to 1450 nm and the light of the first overtone region from 1450 to 1900 nm. The decrease in light intensity or increase in absorbance in these regions is due to chemical and physical effects including large water absorbance bands at 1450 and 1930 nm. The observed absorbance increases rapidly and then levels off as the shims are removed and the sample probe tip is brought into contact with the sample. Generally, using a highly absorbing sample region allows a sharp contrast in returned intensity as a function of distance between a sample probe tip and a sample site as specular reflectance drops off rapidly with contact and magnitude of photons returning from the sample are limited. For example, in noninvasive glucose concentration estimation, a large drop in observed intensity is observed within the last about 0.1 millimeter before contact of a sample probe tip with a tissue sample site at about 1450 nm. This is confirmed by observing that at all wavelengths the intensity decrease is most significant with this single change in spacer height and indicates that specularly reflected light is significantly reduced and that the resulting spectra are now dominated by the absorbance and scattering nature of the tissue sample. Subsequent removal of spacers results in a further displacement of the tissue sample by the sample probe. Increasing displacement of the tissue sample by the sample probe result in changes in the observed absorbance of spectral bands associated with chemical and physical features. For example, two large water absorbance bands are observed centered at 1450 and 1930 nm. Smaller fat and protein absorbance bands are observed in the first and second overtone spectral regions. Scattering effects are observed throughout the spectrum but are most prevalent in the higher energy region of the spectra. The sample collected with the 1.2 mm spacer that resulted in insufficient contact of the sample probe with the tissue sample results in artificially low absorbance across the spectrum due to the collection of spectrally reflected light into the collection optics of the sample probe. In order to enhance the chemical features observed in the first and second overtone spectral windows, the spectra were first smoothed across time and subsequently smoothed across wavelengths with a Savitsky-Golay 13 point second derivative. The resulting spectra are presented in FIG. 3. The second derivative reduces the scattering characteristics and allow the observation of the chemical features. The spectral minima observed at 1152, 1687, and 1720 nm are dominated by the absorbance of water, protein, and fat, respectively.

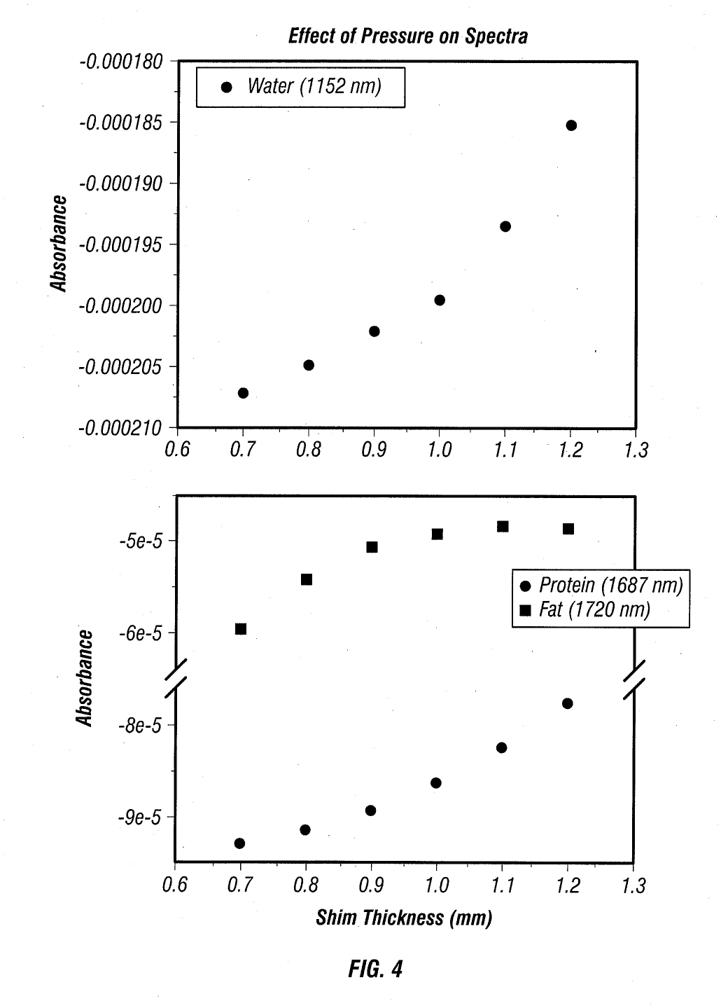

The change in absorbance of the water, protein, and fat spectral features is plotted as a function of displacement in FIG. 4. In this example, the absorbance of all three chemical features is observed to decrease with increasing displacement of the sample probe into the tissue sample through the use of smaller shims. The dependence of the absorbance of the individual chemical and physical features as a function of tissue displacement is dependent upon a range of factors. The factors include: the physical dimension of the sample probe tip interfacing with the tissue sample, the dimension of the aperture in the guide, the chemical composition of the tissue sample, the rate of displacement of the sample probe into the tissue, and a historesis effect of previous contact of an outside object on the sample site.

The displacement of the tissue sample by the sample probe results in compression of the sample site. The displacement results in a number of changes including at least one of:
a change in the localized water concentration as fluid being displaced;
a change in the localized concentration of chemicals that are not displaced such as collagen; and
a correlated change in the localized scattering concentration.

In addition, physical features of the sample site are changed. These changes include at least one of:
compression of the epidermal ridge;
compression of the dermal papilla;
compression of blood capillaries;
deformation of skin collagen; and
relative movement of components embedded in skin.

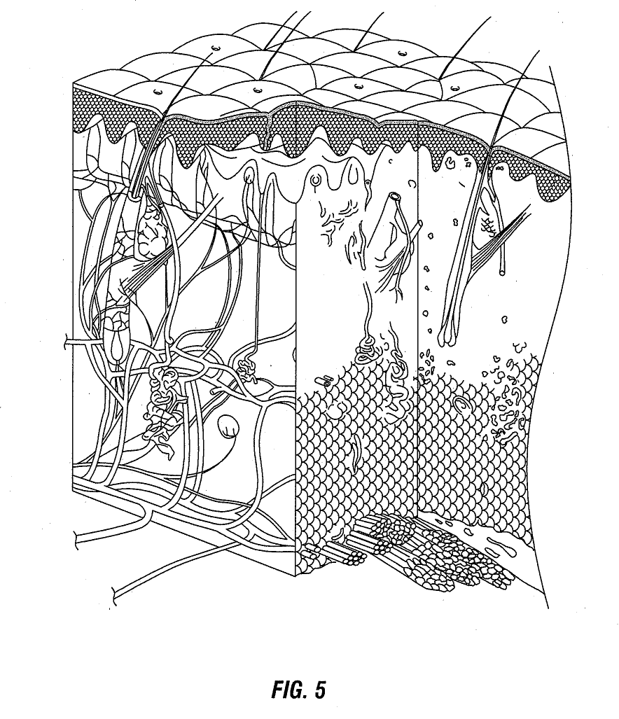

In this example, chemical and physical changes are observed with displacement of the sample probe into the tissue sample. Specific chemical features at three wavelengths are described. However, the displacement of tissue is demonstrated by this example to effect the spectra over a wide range of wavelengths from 1100 to 1930 nm. Additional spectral data shows these pressure effects to be present in at least the infrared region extending out to 2500 nm. Further, the displacement effects are described for a few particular chemical and physical structures. The displacement of tissue also effects a number of additional chemical, physical, and structural features of skin presented in FIG. 5.

Tissue Displacement Control

Displacement of the tissue sample by the sample probe results in changes in noninvasive spectra. Displacement of the sample tissue is related to pressure applied to the sample tissue. However, as the tissue is deformed the return force applied by the tissue sample to the sample probe varies. Therefore, it is preferable to discuss the sample/tissue interaction in terms of displacement instead of pressure.

Displacement of the tissue sample by the sample probe is preferably controlled between an insufficient and excessive displacement or pressure. Insufficient contact of the sample probe with the tissue sample is detrimental. The surface of the skin tends to be rough and irregular. Insufficient contact results in a surface reflection. Contact between the sample probe and the tissue sample minimizes air pockets and reduces optical interface reflections that contain no useful information. Contact or close proximity of the sample probe tip to the sample site preferably provides good optical transmission of source illumination into the capillary layer where the analytical signal exists while minimizing reflections from the surface of the skin that manifest as noise. Excessive displacement of the tissue sample by the sample probe is detrimental. For noninvasive glucose concentration determination using near-infrared spectroscopy, the primary region of interest for measurement of blood borne analytes is the capillary bed of the dermis region, which is approximately 0.1 to 0.4 mm beneath the surface. The capillary bed is a compressible region and is sensitive to pressure, torque, and deformation effects. The accurate representation of blood borne analytes that are used by the body through time, such as glucose, relies on the transport of blood to and from the capillary bed, so it is not preferable to restrict this fluid movement. Therefore, contact pressure/displacement is preferably minimal so as to not excessively restrict or to partially restrict for an extended period of time flow of blood and interstitial fluids to the sampled tissue region.

Analyzer

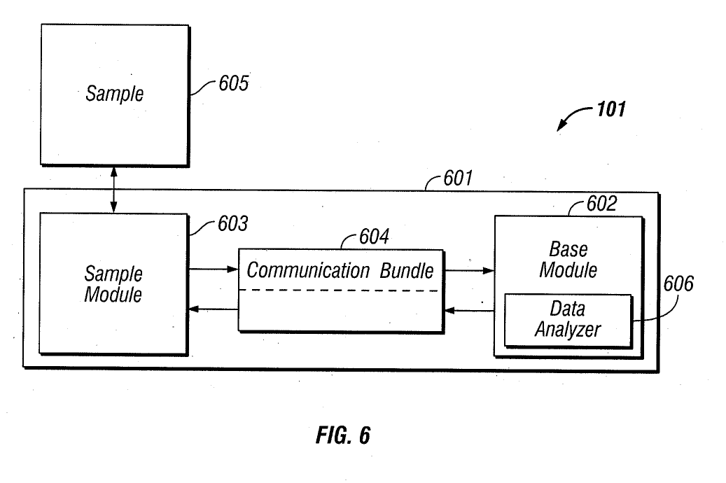

Referring now to FIG. 6, a noninvasive analyzer 101 is represented. As described, supra, in one embodiment the analyzer 101 contains a base module 602 interfaced to a sample module 603. The base module is any of:
integrated with the sample module;
physically coupled to the sample module through a communication bundle 604; and
physically separated from the sample module 603 and coupled with the sample module through wireless communication.

The sample module interfaces with a sample 605. An data analyzer 606 for analyzing collected data, such as spectra and subject data, preferably is contained within or coupled to the base module 602.

Capacitive Probe Control

While many different types of sensors may be used, capacitance sensors or touch sensors are particularly useful for determining any of:
tilt of a sample probe relative to a sample site;
distance of a sample probe tip to a sample site;
x,y-position of a sample probe tip relative to a sample site;
relative distance of a sample probe tip to a sample site; and
contact of a sample probe tip with a sample site.

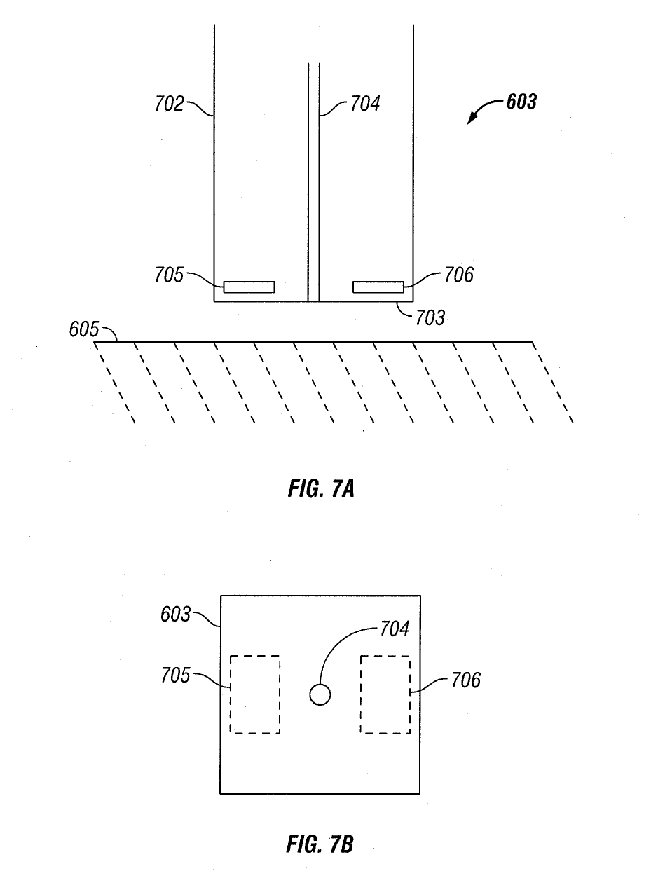

Referring now to FIG. 7, FIG. 7A is a side view of a sample module 603 having a sample probe 702 having a sample probe tip 703 illustrated relative to a tissue sample or sample site 605. The sample probe 702 is illustrated with an optional collection optic 704, and two halves of two separate capacitance conductors 705, 706. An end view of the exemplary sample probe 702 is illustrated in FIG. 7B.

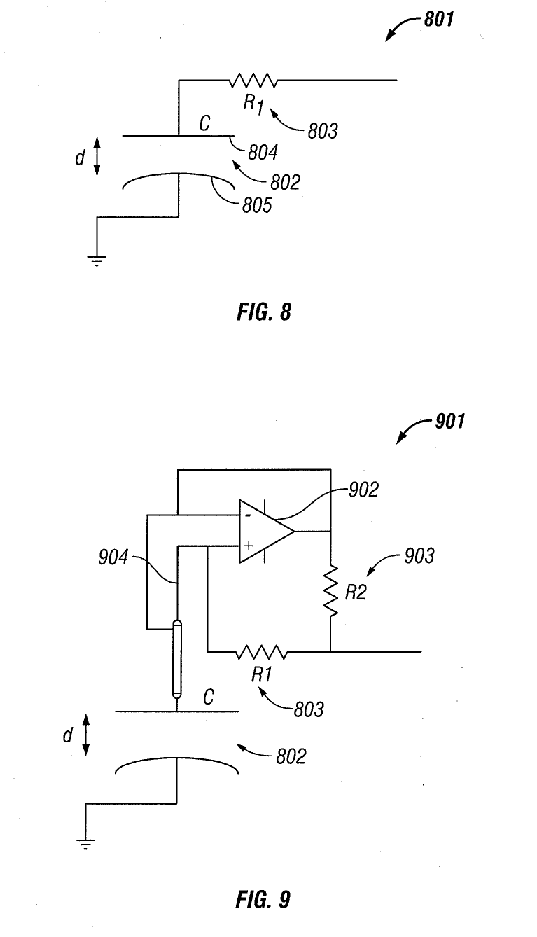

Referring now to FIG. 8, a circuit 801 used to determine proximity of a subject's sample site to the sample probe is presented. The circuit includes a capacitor 802 and a first resistor 803. Here the capacitance, C, is calculated according to equation 1

$$C ∝ \frac{A}{d} \qquad ( 1 )$$

where capacitance, C, is proportional to the area, A, of the capacitor divided by the distance, d, between the capacitor plates. The capacitor has two plates. The first capacitor plate 804 is integrated or connected to the sample module, preferably the sample module sample probe tip. The second capacitor plate 805 is the deformable material, such as a skin sample, body part, or a tissue sample site. The assumption is that the person is a capacitor. A typical adult has a capacitance of about 120 pF. Capacitance of different people will vary. The time constant of the capacitor/resistor in FIG. 8 is calculated according to equation 2
T=RC (2)
where the time constant, T, is equal to the resistance, R, times the capacitance, C. Hence, the distance between the capacitor plates is calculated through the combination of equations 1 and 2 through the measurement of the circuit time constant. For example, the time constant is the time required to trip a set voltage level, such as about 2.2 volts, given a power supply of known power, such as about 3.3 volts. The time constant is used to calculate the capacitance using equation 2. The capacitance is then used to calculate the distance or relative distance through equation 1. For example, as a distance between a sample site, such as a forearm or digit of a hand, and the capacitor plate decreases, the time constant increases and the capacitance increases. The measure of distance is used in positioning the probe at or in proximate contact with the sample site without disturbing the sample site.

Referring now to FIG. 9, an example of an enhancement of the circuit in FIG. 8 is presented. A second circuit 901 using the capacitance 802/resistor 803 combination of the first circuit is enhanced to amplify the signal to noise using an operational amplifier 902 and a second resistor 903. The gain of the circuit is the ratio of the first resistor 803 and the second resistor 903. Optionally, the voltage between the wire leading to the operation amplifier and a surrounding tube/shell 904 is held at or about zero to minimize parasitic capacitance of the long wire and/or to minimize changes in capacitance caused by changes in the environment. A microprocessor used to measure the time constant is attached to any of the circuits herein described.

In use, the distance or relative distance between the sample probe tip and the sample site is determined using the capacitive signal and a calibration. The calibration is generated by moving the capacitive sensors over a known distance while collecting raw capacitance signals and determining a correlation or fit between the sample readings and the known distances.

In one example, the distance between the sample probe tip and the sample is determined and controlled so as to not displace the localized sample site skin/tissue which, as described supra, can lead to degradation of the sample integrity in terms of collected signal-to-noise ratios and/or sampling precision. Examples are used to illustrate the use of the capacitance sensor in the context of a noninvasive analyte property determination.

In another example, the distance or relative distance between the sample probe tip and the sample site is determined using a single capacitor. The sample probe is brought into close proximity with the sample site using the time constant/distance measurement as a metric. In this manner, the sample probe is brought into close proximity to the sample site without displacing the sample site. Due to the inverse relationship between capacitance and distance, the sensitivity to distance between the sample site and the sample probe increases as the distance between the sample and probe decreases. The distance between the sample site and the tip of the sample probe is readily brought to a distance of less than about one millimeter. Capacitance sensors, as used herein, are readily used to place the sample probe tip with a distance of less than about 0.1 millimeter to the sample site. In this example, multiple capacitors are optionally used to yield more than one distance reading between the sample probe tip and the sample site. Multiple capacitive sensors are optionally used to control tilt of the sample probe tip along x- and/or y-axes.

Sample Probe Orientation Control

Two or more capacitance sensors are optionally used for leveling the tip of the sample probe relative to the morphology of the sample site. The distance between the sample site and the probe tip is measured using two or more capacitor plate pairs. Illustratively, each capacitor may be within a suitable circuit such as shown in, for example, FIG. 9 shows a first capacitor plate in the sample probe tip and a second capacitor plate, such as skin tissue of a human. If one capacitor reads a larger distance to the sample site than the second capacitor, then the probe tip is moved to level the probe by moving the larger distance side toward the sample, the smaller distance side away from the sample, or both. The sample probe tip tilt or angle is either moved manually or by mechanical means.

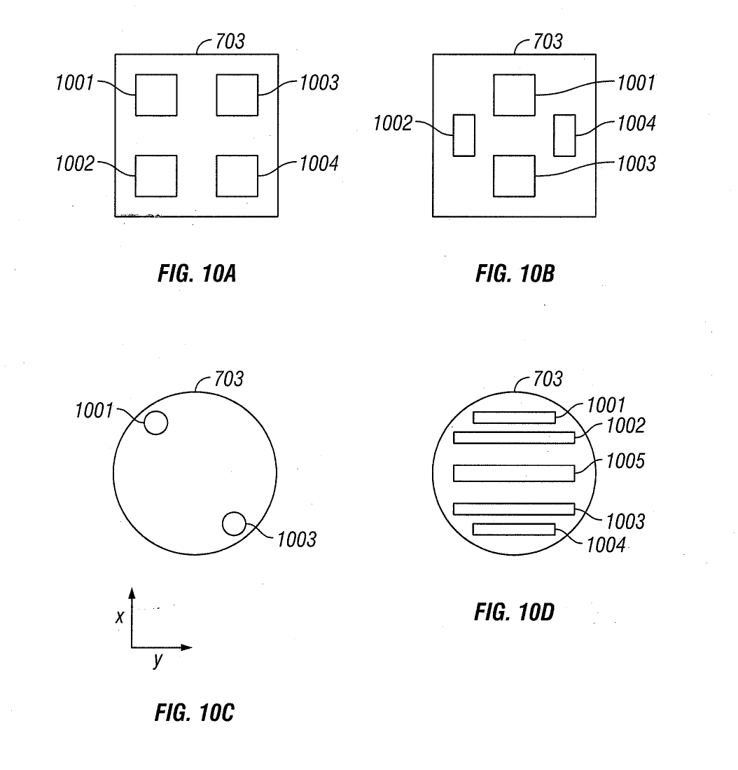

Referring now to FIG. 10, exemplary capacitance layouts on the tip of a sample probe are provided. Referring now to FIG. 10A, four capacitance sensors 1001-1004 are used allowing for detection of tilt along the x-, y-, or x and y-axis by comparison of signal strength between two or more of the four sensors 1001-1004. Alternatively, signals from two or more capacitors are combined, such as via a summation or an addition. For example, signals from sensors 1001 and 1002 are combined and compared against the combined signals of sensors 1003 and 1004. The combined signals are used as a feedback to a controller controlling tilt, such as along the y-axis. Referring now to FIG. 10B, four capacitance based sensors are placed at or near the surface of a probe tip 703. In this example, the sensors are placed along the x- and y-axis for ease in determining x- and y-axis level of the sensor probe. Referring now to FIG. 10C, two capacitance sensors 1001, 1003 are shown off axis on a round sample probe tip 703. Referring now to FIG. 10D, five capacitance sensors 1001-1005 are shown that vary in position along a single direction, the x-axis as shown. As orientated, the five sensors are used to detect tilt of the sample probe relative to the sample site and/or proximity of the sample probe tip to the sample site. Generally, any number of capacitance sensors are used in any geometric configuration on, near, or within a sample probe tip of any geometry.

Capacitive sensors have multiple advantages including: cost, response time, weight, size, and sensitivity. In addition, for biomedical applications, the capacitance sensors are optionally embedded into the sample probe tip providing a dielectric barrier between the sensor and the subject, which is beneficial in terms of safety requirements, such as government regulatory requirements.

Sample Probe Movement

Movement of the sample probe is preferably performed using an electro-mechanical converter. After an initial command input, the actuator system 102 is preferably controlled using a controller 105 provided with signal from the probe system sensor 104 as illustrated in FIG. 1. For example, if a capacitance sensor indicates that the probe tip is too far from the sample, an electro-mechanical converter is used to translate the sample probe along the z-axis toward the sample site. Similarly, if two capacitance signals from opposite sides of the sample probe tip indicate that the sample probe tip is not normal to the sample site, then one or more actuators are used to adjust the tilt of the sample probe or the level of the sample probe tip relative to the sample site surface. In one case, one or more sensors are used in combination with one or more actuators to control sample probe position in terms of any of:
x-position;
y-position;
z-position;
tilt; and
rotation.

In one illustrative system, one or more capacitance signals from one or more capacitance pairs generate signals to a controller. The controller uses the capacitance signal to direct at least one actuator to position a sample probe tip of an analyzer into proximate contact with a tissue sample site. In one case, the sample probe tip is brought into contact with the tissue sample with minimal or no displacement of the tissue sample site by the sample probe tip. In a second case, the sample probe tip is further displaced into the tissue sample for a distance of less than about one millimeter, and preferably a distance of less than one-half of one millimeter.

A capacitance sensor is used to exemplify the invention. Generally, any sensor capable of sensing distance between the sample site or skin surface and a portion of the optical probe is usable in adjusting any of distance and tilt. Examples of sensors used for this purpose include magnetic, optical, current, inductive, ultrasonic, resistive and electrical contact based sensors.

Additional mechanisms for adjusting the sample-probe orientation relative to the tissue sample include use of lead screws to force the sample probe to a given orientation or the use of a rotating shim of tapered thickness. The rotating shim rotates relative to the sample probe tip. The shim is preferably connected to the sample probe by a mechanical means such that as the shim rotates, the sample probe is driven up or down.

Optical Sensors

An optical sensor is optionally used to detect distance between an analyzer and a sample, tilt of a sample probe relative to a sample, and/or contact or proximate contact of a sample probe tip with a sample site. The optical sensors are optionally configured in the orientation of any of the above described orientations of the capacitance proximity sensors, such as at or near the edges of the sample probe tip or in close proximity to a collection optic or fiber. Each of the optical sensors includes an illumination source and a detector. The source is optionally independent for each sensor, such as through one or more light emitting diodes. Alternatively, two of more of the sensors share a common source, such as a single light emitting diode or an incandescent source, such as a tungsten halogen lamp. The detectors are optionally placed within the sample probe tip, near the sample probe tip, or at a distance from the sample probe tip. In the later case, light is coupled to the detector via optics, such as fiber optics.

The optical sensors are preferably used to detect contact or near contact of the sample probe tip with the sample through the use of over absorbed wavelengths in human tissue, such as about 1450 nm where the surface reflection intensity dominates over signal returning via scattering from within the tissue sample. In one mode, the sample probe is moved down the z-axis until the specular light approaches zero. Movement of the sample probe is as described using the capacitance sensor and includes movement under manual control or automated control with or without the use of one or more actuators and with or without a feedback sensor and controller.

In a second mode, as surface reflection intensity is known to be a function of distance, the surface intensities from two or more optical sensors are compared in order to determine relative tilt of the sample probe relative to the tissue sample and the information used to adjust tilt until near tangential contact is achieved. In yet another mode, the optical signal, from the source of the analyzer that is detected by the detector used to generate a signal for subsequent an analyte property determination, is used to determine proximity to or contact of the sample probe tip with the sample site. For instance, the spectrum determined using photons gathered by the collection fiber 704 is used to determine proximity of or contact of the sample probe with the tissue sample site.

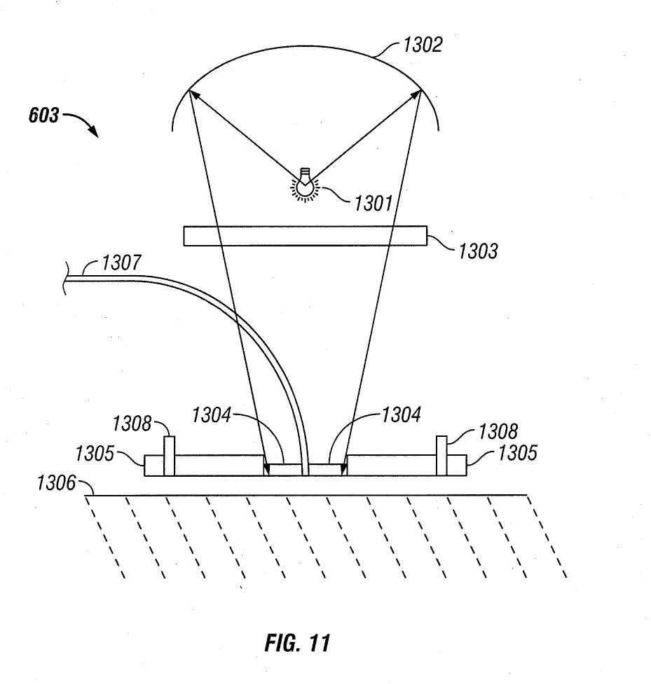

Referring now to FIG. 11, an illustrative example of an optical probe is provided. In this example, portions of a sample probe 603 are pictured. In this example, photons from a source 1301 are reflected off of a backreflector 1302 through a first optic 1303 and through a second optic 1304 to a sample 1306, such as skin. The first optic is optionally used to remove unwanted emanating photons from the source that would otherwise optically heat the sample. The second optic passes light and is optionally used to stabilize any of: sample site topology, sample site hydration, and to mechanically stabilize or locate a collection optic 1307, such as a fiber optic. In this example, detectors 1308 are used to detect light from the sensor source 1301 in order to control tilt of the sample probe relative to the sample and/or distance between the tip of the fiber optic 1305 and the sample 1306.

In another illustrative system, inductive signals are used to determine proximity of the sample site to the tip of the sample probe.

Conductance Sensor/Conductance Probe Control

In another illustrative system, completion of an electrical circuit is monitored in order to determine contact between a tip of a sample probe of an analyzer and a skin tissue sample. Electrical contact sensors are further described in U.S. provisional patent application No. 60/864,375 filed Nov. 3, 2006, which is incorporated herein in its entirety by this reference thereto.

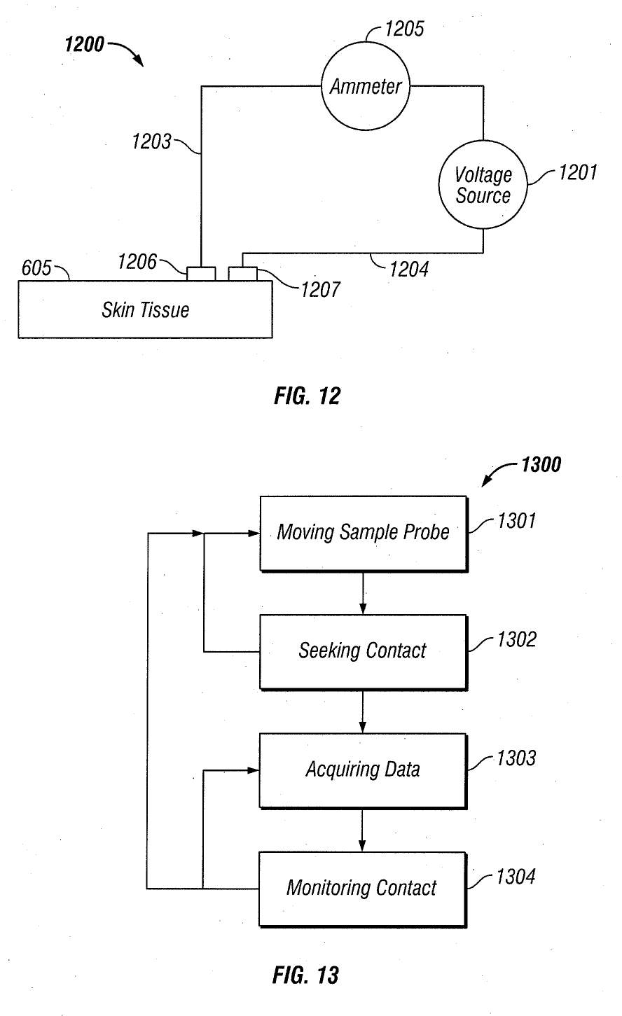

Referring now to FIG. 12, a conductive contact sensing system 1200 is used that includes at least a power supply 1201, such as a voltage source, replaceably interfaced to a skin tissue sample 605. The voltage source is preferably a low voltage source, such as, a source providing about 1, 3, 6, or 12 volts. The voltage is converted to a current carried over one or more electrical conduits 1203, 1204 through an power meter 1205, such as an ammeter. In this example, the circuit is initially open and is closed when the electrical conduits 1203, 1204 are brought into contact with the skin tissue 605, such as through one or more connections 1206, 1207. For example, current flows from the power source 1201 to the skin through a first electrical conduit 1203 and through a contacting element 1206, such as a tip of a sample probe of an analyzer. In a first case, current returns to the voltage source via a separate electrical conduit 1204, such as a conducting line. In this case, the first and second contacting elements 1206, 1207 are optionally contained in one housing or in two housings. In a second case, the current entering the skin tissue sample 1202 completes an electrical circuit by passing to ground from the skin, the power supply also being grounded. In use, a change in current is observed when the circuit is completed. For example, the circuit is completed when the relative distance between the contacting element 1206 and skin tissue 605 is reduced to a contacting distance of less than about 0.1 millimeter and typically to a distance of less than about 0.01 millimeters.

A second example of a conductive contacting system is provided. In this example, the power supply, such as a bias voltage of about six volts, is an input to an operational amplifier. The voltage is converted to current across a resistor, such as resistor of about 100 MOhm resistance. When the circuit is completed by contacting the arm, the current flows into the arm. The decreased resistance to current flow upon contact with the arm results in an increase in the observed signal. Preferably, the observed current is compared with the bias current using a differential amplifier and the resulting signal is converted to a voltage at an analog to digital converter. Optionally, the bias current is filtered using a low pass filter, such as a 2.5 Hertz low pass filter.

Referring now to FIG. 13, a flowchart of an example of use of a sample probe 702 is provided. Initially, tissue movement of the sample relative to the sample probe is performed. The sample probe is moved (block 1301) and contact between the sample probe and tissue sample is sought (block 1302) using a contact sensor, such as any described herein. This process is iterative or the sensing signal is sought while the sample probe is moved. Upon contact, acquisition of data commences. Alternatively, data is additionally acquired as contact between the sample probe and tissue sample is sought. At this point, the sample probe:
continues toward the sample and minimally displaces tissue volume;
stops;
in position controlled using a contact sensing feedback signal; and
is iterative backed away and toward the tissue providing reestablished contact.

For example, a contact qualification monitoring system is used to determine contact or proximate contact of the sensor system with a tissue sample. The conductive sensor provides a feedback signal to a controlling algorithm or actuator telling the analyzer when to stop moving the sample probe. Spectral data is acquired (block 1303) during the contact sensing process and/or after contact is established. The contact sensing process is also referred to herein as a hunt process. Optionally, once contact is determined, the conductive sensor system acts as a system for monitoring continued contact (block 1304). If contact is lost, the analyzer is preferably directed to reestablish contact between the optical probe and the tissue sample. Data acquisition is suspended until contact is reestablished. The spectral data acquisition system, conductive contact determination/monitoring system, and actuator movement system are optionally used serially, iteratively, and/or in parallel with each other.

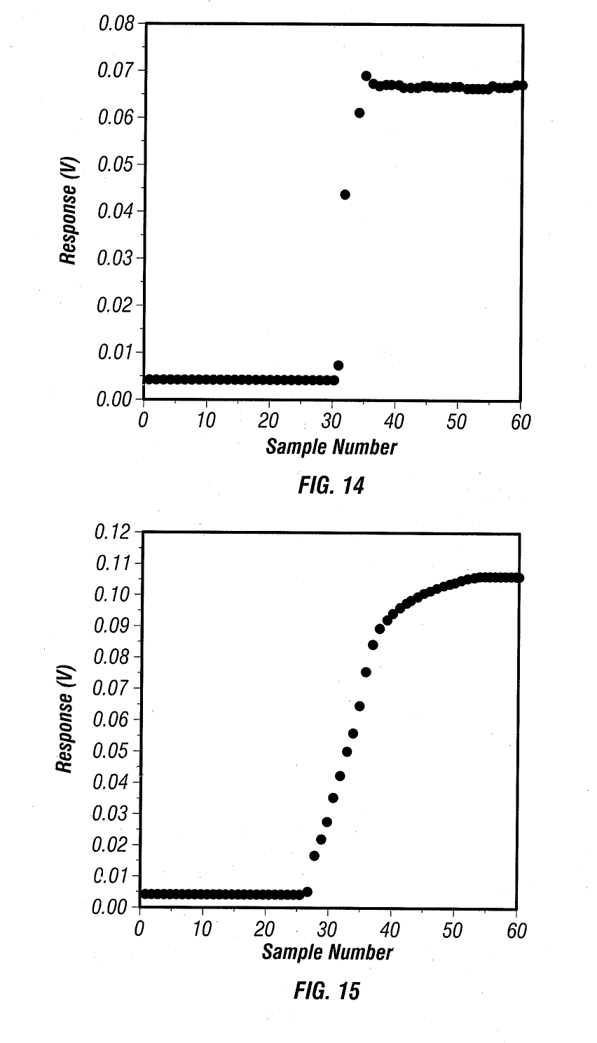

Referring now to FIG. 14, a current monitored contact event between an analyzer and a skin tissue sample is demonstrated. In this example, a sample probe of an analyzer is moved toward a skin tissue sample as a function of time. A response of a conductive contact sensor is provided as the sample probe moves. Initially, a bias voltage from an analog to digital converter is obtained, which is nominally flat as a function of time. As contact is achieved between the tip of the sample probe and the skin tissue, the observed voltage rises rapidly to a new level. Once full contact is made, the returned voltage stabilizes at a new level. Both the slope of the rising response curve and the difference between the initial and final observed voltages are indicative of the contacting event. The high slope between the bias and stabilized voltage is indicative of the contact event occurring over a short time period. The difference is indicative of the quality of the contact and also of the state of the skin tissue. For example, a fully hydrated skin sample will yield a larger difference than a poorly hydrated skin sample. The current magnitude also provides information about the contact event, such as a coupling fluid thickness between the skin and the probe. For instance, since FC-40 has a high resistance, the observed contact current increases as the thickness of the FC40 layer on the skin decreases.

Referring now to FIG. 15, another example of a sample probe having both an optical sampling system and a electrical contact monitoring system is provided. FIG. 15 illustrates the conductive contact event as a tip of the sample probe is moved into contact with skin tissue. In this example, the slope of the rising response curve is illustrative of conductive contact being made over a longer time period, such as occurs when the skin sample stretches as the tip of the sample probe hits the skin surface. As a result, the sample probe further displaces the tissue before a stabilized contact is obtained. Selection of later optical samples collected when the electrical contact is stabilizing with acceptable contact allows calibration and/or prediction using the optical samples where good contact between the sample probe and skin tissue sample is present. This process yields samples with smaller changes in optically sampled tissue volume as a function of time. The selection of samples based upon the electrical contact sensor response thus leads to selection of prediction samples bounded by calibration data, thereby leading to robust, accurate, and precise analyte property estimations. The use of the conductive contact response in selection of corresponding optical samples for use in calibration and/or prediction is beneficial. For instance, only optical samples having full contact are selected for calibration and/or prediction. Alternatively, samples are selected having any contact, such as samples having a conductive contact response greater than the initial bias voltage plus two times the noise of the initial non-contacting samples.

The use of conductive contact is also useful data in determining validity of an optical contact metric.

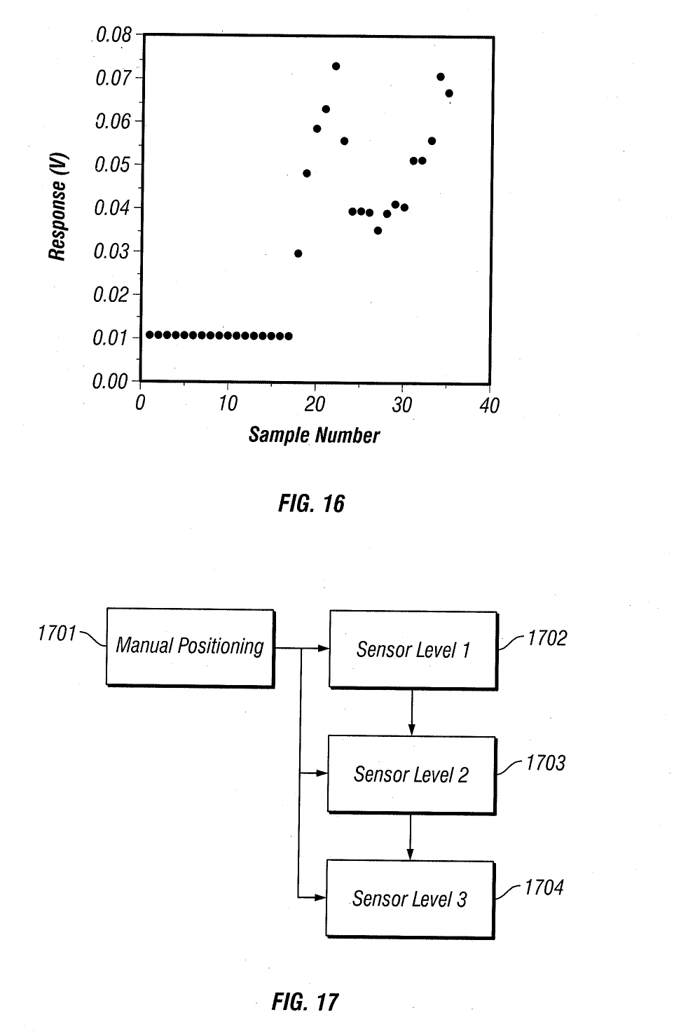

Referring now to FIG. 16, yet another example of a conductive contact sensor response is provided. In this example, the tip of an optical sample probe, configured with a conductive contact sensor, is moved toward a skin tissue sample. Initially, no contact is made. At sample number eighteen initial contact is made; full contact is made within a few additional samples. The observed voltage response of the conductive contact sensor subsequently fell due to loss of contact. Falling contact voltages result from a number of scenarios, such as the skin slipping or sagging relative to the tip of the sample probe. Subsequently, the sample probe tip was repositioned and the conductive contact sensor indicates when contact is reestablished after moving the probe, such as by monitoring the response voltage until movement of the sample probe brings the response back up to a contact level meeting specification or to a full contact level. In this case, the sample probe was moved toward the skin to regain full contact.

In yet another example, a current-measurement contact sensor is placed adjacent to or surrounding an optical detection fiber. A metal tube can surround the optical fiber and the contact sensor uses the tube for its measurement interface. When the metal tube shows contact, it is determined that the tip of optical collection means, such as the end of a fiber optic, is also making contact and the measurement may be made. The tip of the sample probe is preferably driven into the tissue sample along the z-axis a small amount, such as about 25 to 200 μm, in order to ensure the probe is in contact with the skin throughout the duration of the measurement.

In yet another example, sensors are placed at away from the center of the tip of the sample probe. The circumferentially distribute contact sensors are used to indicate when the probe comes down at an angle and a corner of the tip of the sample probe contacts the skin tissue before the center of the sample probe. These contact sensors are optionally in a ring that indicates contact at any point around the probe or are sensors that indicate contact at specific locations, such as at corners or edges of the tip sample probe. Alternatively, sensors are placed along each of the axial, y-axis, and longitudinal axis, x-axis of a body part so that the phasing of the sensors contacting in time may be analyzed to help interpret the geometry changes occurring during the contact event. A single circuit is preferably multiplexed with multiple sensors to minimize the amount of additional cabling required with additional sensors.

Preferably, a current reading from a given sensor is be taken before contact to establish a baseline. The current is periodically monitored during a measurement sweep and more frequently as the probe approaches the arm in order to prevent a crash into skin due the probe stopping too late. In a real-time hunting scenario, such as samples collected at about five Hz as the probe approaches the arm, the contact-current is compared to the intensity at about 1290 or about 1450 nm returning from the arm or to capacitance measurements to assess the approach of the probe to the arm and slow its descent as necessary.

In yet another embodiment of the invention, a conductive sensor around the perimeter of the optical detection fiber is used to identify measurements and scans within a dynamic measurement that have intimate contact between the skin and the optical detection fiber. Such a sensor is used to control the dynamics of probe contact and/or to select the scans with the most intimate contact for use in calibration and for qualifying prediction spectra.

Multiple Sensors

In still yet another embodiment of the invention, two or more signals or signal types are used in the process of positioning a sample probe tip relative to a sample site. For example a first signal is used to position the sample probe coarsely relative to the sample site and a second sensor is used to control sample probe positioning at a closer distance to the sample site. Optionally, a third sensor is used to still more finely position the sample probe tip relative to the sample site.

Referring now to FIG. 17, after manual positioning the sample probe, even at instrument setup, positioning is performed using just the third sensor, the second and third sensor, or all three sensors. Each of the sensors provides a distinct signal. For instance, one sensor is based upon a capacitance reading while a second is generated from an optical or conductance signal. Different sensors are used for different levels. Generally as positioning goes from gross alignment to fine alignment:
distance between a sample probe tip and a sample site generally decreases;
optionally different sensors are used as input at different distances between the sample probe tip and the sample site;
preferably precision of positioning increases; and
preferably different sensors or associated positioning algorithms are used at various levels.

This system allows for various sensors that are best used to estimate position of the sample probe at different distances from the sample site to be used serially or in a parallel fashion. For example, a first sensor is used for coarse alignment. Examples include:
machine vision;
a targeting system; and
use of a fluorescence tag.

Generally, the first sensor is used to bring the tip of the sample probe into a coarse position relative to the sample site. The first sensor is for example used to move the sample probe in the range of about 0.2 to 10+ mm from the sample site. A second sensor is optionally used with or without a first sensor. The second sensor provides a signal distinct from the signal from the first sensor. The second sensor is used to estimate distances of the sample probe tip to the sample site that are generally smaller than the first sensor, described supra. For example, the second sensor is used in positioning the sample probe in a range of about 0 to 1 mm from the sample site or ranges therein. An example of a second sensor is a capacitance sensor, as described herein.

In yet another example, a sample probe is placed near a sample site. Initially, a capacitive sensor provides a time constant to the controller. The controller uses the time constant to determine distance between the tip of the sample probe and the sample site. The controller then sends a signal to one or more actuators that reposition the sample probe. Typically, the first process is iterated and the sample probe tip is moved toward the sample site. As the tip of the sample probe approaches the sample site, two or more capacitive sensors yield corresponding time constants that are used by the controller to determine the tilt of the sample probe relative to the surface of the sample site. The controller sends directions to one or more actuators to adjust the sample probe. Typically, this second iterative process is repeated until the surface of the sample probe tip is about parallel to the sample site surface. One or both of the first and second iterative processes are repeated either sequentially or in parallel. A third optical sensor or a conductance sensor yields a signal interpreted by the controller to yield a decision about fine movement of the sample probe relative to the sample site, typically in terms of z-axis position as tilt was previously adjusted. This process iteratively uses optical signal to finely position the sample probe tip in contact with or in proximate contact with the sample site surface.

Herein, the first process uses sensor 1 to control z-axis position; the second process uses sensor(s) 2 to control the tilt of the sample probe relative to the tissue sample site; and the third process uses sensor 3 to finely position the sample probe tip into proximate contact or contact with the sample site.

In still yet another example, a targeting system is used to initially position a sensor relative to the sample site, such as in the x- and y-axes. A second sensor is then used to adjust the probe, such as in tilt of the sample probe relative to a sample site and/or distance between the sample probe tip and the sample site. Optionally, a third sensor is used to finely control the sample probe tip, such as to proximate contact with, initial contact to, and/or displacement into a sample site.

A targeting system is used to position a measuring system, such as an optical probe of a noninvasive glucose concentration analyzer. A targeting system targets a tissue area or volume of a sample. For example, a targeting system targets a surface feature, one or more volumes or layers, and/or an underlying feature, such as a capillary or blood vessel. The measuring system preferably contains a sample probe, which is optionally separate from or integrated into the targeting system. The sample probe of the measuring system is preferably directed to the targeted region or to a location relative to the targeted region either while the targeting system is active or subsequent to targeting. A controller is used to direct the movement of the sample probe in at least one of the x-, y-, and z-axes via one or more actuators. Optionally the controller directs a part of the analyzer that changes the observed tissue sample in terms of surface area or volume. The controller communicates with the targeting system, measuring system, and/or controller.

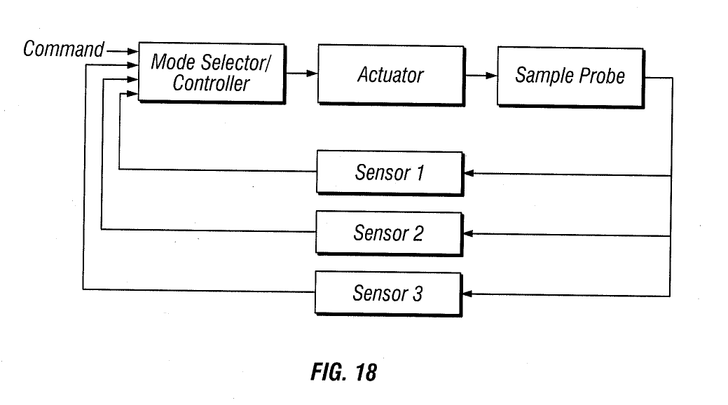

Generally, a mode selector or controller takes as input a command or input from one or more sensors and subsequently directs an actuator to move a sample probe as shown in FIG. 18.

Targeting System

There exist a large number of targeting and measuring system configurations. Some features of illustrative configurations are outlined here. The targeting system and measuring system optionally use a single source that is shared or have separate sources. The targeting is optionally used to first target a region and the measuring system subsequently samples at or near the targeted region. Alternatively, the targeting and measuring system are used over the same period of time so that targeting is active during sampling by the measuring system. The targeting system and measuring system optionally share optics and/or probe the same tissue area and/or volume. Alternatively, the targeting and measuring system use separate optics and/or probe different or overlapping tissue volumes. In various configurations, neither, one, or both of the targeting system and measuring system are brought into contact with the skin tissue at or about the sample site. Finally, permutations and combinations of the strategies and components of the embodiments presented herein are possible.

The targeting system targets a target. Targets include any of:
a natural tissue component;
a chemical feature;
a physical feature;
an abstract feature;
a marking feature added to the skin;
a skin surface feature;
a measurement of tissue strain;
tissue morphology;
a target below the skin surface;
a manmade target;
a fluorescent marker;
a subcutaneous feature;
a dermis thickness within a specification;
capillary beds;
a capillary;
a blood vessel; and
arterial anastomoses.

Examples of marking features added to the skin include a tattoo, one or more dyes, one or more reflectors, a crosshair marking, and positional markers, such as one or more dots or lines. Examples of a skin surface feature include a wart, hair follicle, hair, freckle, wrinkle, and gland. Tissue morphology includes surface shape of the skin, such as curvature and flatness. Examples of specifications for a dermis thickness include a minimal thickness and a maximum depth. For example, the target is a volume of skin wherein the analyte, such as glucose, concentration is higher. In this example, the measuring system is directed to image photons at a depth of the enhanced analyte concentration.

A targeting system typically includes a controller, an actuator, and a sample probe. Examples of targeting systems include a planarity detection system, optical coherence tomography (OCT), a proximity detector and/or targeting system, an imaging system, a two-detector system, and a single detector system. Examples of targeting system technology include impedence, acoustic signature, ultrasound, use of a pulsed laser to detect and determine distance, and the use of an electromagnetic field, such as radar and high frequency radio-frequency waves. Sources of the targeting system include a laser scanner, ultrasound, and light, such as ultraviolet, visible, near-infrared, mid-infrared, and far-infrared light. Detectors of the targeting system are optionally a single element, a two detector system, an imaging system, or a detector array, such as a charge coupled detector (CCD) or charge injection device or detector (CID). One use of a targeting system is to control movement of a sample probe to a sampling location. A second example of use of a targeting system is to make its own measurement. A third use is as a primary or secondary outlier detection determination. In its broadest sense, one or more targeting systems are used in conjunction with or independently from a measurement system.

Different targeting techniques have different benefits. As a first example, mid-infrared light samples surface features to the exclusion of features at a depth due to the large absorbance of water in the mid-infrared. A second example uses the therapeutic window in the near-infrared to image a feature at a depth within tissue due to the light penetration ability from 700 to 1100 nm. Additional examples are targeting with light from about 1100 to 1450, about 1450 to 1900, and/or about 1900 to 2500 nm, which have progressively shallower penetration depths of about 10, 5, and 2 mm in tissue, respectively. A further example is use of visible light for targeting or imaging greater depths, such as tens of millimeters. Still an additional example is the use of a Raman targeting system, such as in WIPO international publication number WO 2005/009236 published Feb. 3, 2005, which is incorporated herein in its entirety by this reference thereto. A Raman system is capable of targeting capillaries. Multiple permutations and combinations of optical system components are available for use in a targeting system.

Controller

A controller controls the movement of one or more sample probes via one or more actuators. The controller may be analogue or digital or a combination, and if digital, may include a microcontroller, microprocessor, application specific logic, or other type of processor along with suitable memory and peripheral components. The controller optionally uses an intelligent system for locating the sample site and/or for determining surface morphology. For example, the controller hunts in the x- and y-axes for a spectral signature. In a second example, the controller moves a sample probe via the actuator toward or away from the sample along the z-axis. The controller optionally uses feedback from the targeting system, from the measurement system, or from an outside sensor in a closed-loop mechanism for deciding on targeting probe movement and for sample probe movement. In a third example, the controller optimizes a multivariate response, such as response due to chemical features or physical features. Examples of chemical features include blood/tissue constituents, such as water, protein, collagen, elastin, and fat. Examples of physical features include temperature, pressure, and tissue strain. Combinations of features are used to determine features, such as specular reflectance. For example, specular reflectance is a physical feature optionally measured with a chemical signature, such as water absorbance bands centered at about 1450, 1900, or 2600 nm. Controlled elements include any of the x-, y-, z-axis position of sampling along with rotation or tilt of the sample probe. Also optionally controlled are periods of light launch, intensity of light launch, depth of focus, and surface temperature. In a fourth example, the controller controls elements resulting in pathlength and/or depth of penetration variation. For example, the controller controls an iris, rotating wheel, backreflector, or incident optic, which are each described infra.

A targeting system used in combination with the positioning systems herein is further described in U.S. provisional patent application No. 60/656,727 filed Feb. 25, 2005, which is incorporated herein its entirety by this reference thereto.

In another example, a capacitance based sensor is optionally used to place a guide element onto a skin sample, such as at a level portion of the skin sample.

Additional examples of the invention are any combination and/or permutation of the embodiments, examples, and/or obvious variants of the examples provided herein.

Those skilled in the art will recognize that the present invention may be manifested in a variety of forms other than the specific embodiments described and contemplated herein. Departures in form and detail may be made without departing from the spirit and scope of the present invention. Accordingly, the invention should only be limited by the Claims included below.

1. A method for operating an apparatus for noninvasively determining glucose concentration in a tissue at a sample site, said sample site having an outer surface, comprising the steps of:
generating a first capacitance signal with a first sensor connected to a noninvasive analyzer, wherein said analyzer comprises a sample probe tip;
orientating a tilt of said sample probe tip relative to the outer surface of the sample site using said first signal;
reducing a distance between said sample probe tip and the outer surface of the sample site, wherein said first signal measures said distance;
generating a contact signal with a second sensor connected to said analyzer;
moving either said sample probe tip or the human tissue until said contact signal indicates proximate contact between said sample probe tip and the outer surface of the sample site; and
optically determining said glucose concentration with said analyzer.

2. The method of claim 1, wherein said second sensor comprises a conductance sensor.

3. The method of claim 1, wherein said second sensor comprises an optical signal.

4. The method of claim 1, further comprising the steps of:
generating a targeting signal with an imaging system connected to said analyzer; and
positioning said sample probe tip relative to the sample site based upon said targeting signal, wherein said steps of orientating, reducing, and moving at least follow said step of generating said targeting signal.

5. The method of claim 1, wherein said first sensor comprises a plurality of capacitor plates, wherein said plurality of capacitor plates result in a plurality of capacitance signals.

6. The method of claim 1, further comprising the steps of:
comparing at least two of said plurality of capacitance signals, and determining tilt prior to said step of orientating tilt.

7. The method of claim 1, further comprising the step of:
using a controller to iteratively direct an actuator to move said sample probe tip relative to the tissue sample site, based upon said capacitance signal.

8. The method of claim 7, further comprising the step of:
iteratively directing movement of said sample probe tip relative to the tissue sample site based upon said contact signal.

9-17. (canceled)

18. A method for operating an apparatus for noninvasively determining an analyte property of a tissue at a sample site, comprising the steps of:
generating a capacitance signal with a first sensor connected to a noninvasive analyzer, wherein said analyzer comprises:
a sample probe tip;
a controller; and
an actuator;
positioning said sample probe tip relative to the sample site, wherein said controller direct said actuator based upon said capacitance signal; and
after said step of positioning, noninvasively determining said analyte property with said analyzer.

19. The method of claim 18, further comprising the step of:
iteratively repeating said step of positioning.

20. The method of claim 18, wherein said stop of positioning further comprises the step of orientating tilt of said sample probe tip relative to the sample site.

21. The method of claim 18, wherein said step of positioning further comprises the step of moving an x-, y-position of said sample probe tip relative to the sample site, wherein said x-position defines a position along a body part and said y-position defines a position across the body part.

22. The method of claim 18, wherein said step of positioning further comprises the step of reducing distance between said sample probe tip and the sample site.

23. The method of claim 22, wherein said step of positioning further comprises the step of proximately contacting said sample probe tip with the sample site.

24. The method of claim 23, wherein said step of positioning further comprises the step of displacing said sample probe tip less than about one millimeter into the sample site.

25-31. (canceled)

32. An apparatus for noninvasively determining a glucose concentration of a tissue at a sample site, having an outer surface, said apparatus comprising:
one capacitor plate yielding a capacitance signal representative of distance between said sample probe tip and the sample site, wherein said one capacitor plate is embedded into a sample probe tip of an analyzer,
wherein said analyzer comprises a controller,
wherein said controller uses said distance in directing an actuator to move said sample probe tip relative to the sample site.

33. The apparatus of claim 32, further comprising:
a contact sensor integrated into said analyzer for generation of a contact signal; and
means for determining contact, based upon said contact signal, between said sample probe tip and the tissue sample site.

34-35. (canceled)

36. An apparatus for providing a measurement of glucose concentration in a tissue sample at a sample site, the tissue sample having an outer surface at the sample site, comprising:
an actuator;
a sample probe tip coupled to the actuator, the sample probe tip having a contact surface for contacting the outer surface of the tissue sample and comprising:
a first capacitor plate disposed in the contact surface of the sample probe tip;
a second capacitor plate disposed in the contact surface of the sample probe tip and spaced away from the first capacitor plate;
a contact sensor disposed in the contact surface of the sample probe tip; and
an optical window disposed in the contact surface of the sample probe tip for receiving optical energy from the tissue sample when the contact surface of the sample probe tip is in engagement with the outer surface of the tissue sample at the sample site; and
a controller responsive to signals from the first capacitor plate, the second capacitor plate, and the contact sensor for operating the actuator to bring the sample probe tip into oriented contact with the outer surface of the tissue sample at the sample site.

37. The apparatus of claim 36, further comprising an analysis component comprising means for:
monitoring capacitance between the first and second capacitor plates and the sample site;
bringing the sample probe tip and the sample site into proximity, as a function of the monitored capacitance;
monitoring the contact sensor for proximate contact of the sample probe tip to the outer surface region of the sample site;
identifying proximate contact between the sample probe tip and the outer surface of the sample site, as a function of the monitored proximity; and
detecting the optical energy from the optical window as a function of the identified proximate contact, to optically measure the glucose concentration.

38. A method for aligning a sample probe tip of a noninvasive glucose concentration analyzer with an outer surface region of a sample site of a tissue, comprising the steps of:
monitoring capacitance between the sample probe tip and the sample site;
bringing the sample probe tip and the sample site into proximity, as a function of the monitored capacitance;
monitoring for proximate contact of the sample probe tip to the outer surface region of the sample site;
identifying proximate contact between the sample probe tip and the outer surface of the sample site, as a function of the monitored proximity; and
optically measuring glucose concentration with the glucose concentration analyzer, as a function of the identified proximate contact.

39. The method of claim 38, further comprising orienting the sample probe tip during the bringing step into a normal alignment relative to the outer surface region of the sample site, as a function of the monitored capacitance.

40. The method of claim 38, wherein the proximate contact monitoring step, comprises monitoring a signal from a conductance sensor disposed at the sample probe tip.

41. The method of claim 38, wherein the proximate contact monitoring step comprises monitoring a signal from an optical sensor disposed at the sample probe tip.
